(19) United States
(12) Patent Application Publication
(10) Pub. No.: US 2024/0301102 A1
(43) Pub. Date: Sep. 12, 2024

(54) TYPE I PHOTOINITIATOR FOR CURING SILICONE COMPOSITIONS

(71) Applicants: ELKEM SILICONES FRANCE SAS, LYON (FR); UNIVERSITE DE HAUTE ALSACE, MULHOUSE CÉDEX (FR)

(72) Inventors: Xavier ALLONAS, MULHOUSE (FR); Ahmad IBRAHIM, QUÉBEC (CA); Jean-Marc FRANCES, MEYZIEU (FR); Perrine THEIL, SAINT-PIERRE DE CHANDIEU (FR); Christian MALIVERNEY, SAINT JULIEN SUR BIBOST (FR)

(21) Appl. No.: 18/280,728

(22) PCT Filed: Mar. 10, 2022
(86) PCT No.: PCT/FR2022/050430

§ 371 Date: Sep. 7, 2023

(30) Foreign Application Priority Data
Mar. 10, 2021 (FR) FR21 02359

(51) Int. Cl. C08F 130/08; C07C 69/24; C08J 3/28; C08K 5/101; C09D 143/04

(52) U.S. Cl. CPC C08F 130/08; C07C 69/24; C08J 3/28; C08K 5/101; C09D 143/04; C08J 2383/04; C08J 2483/04

(57) ABSTRACT

The invention relates to a type I photoinitiator for the free-radical curing of radiation-curable compositions. In particular, the invention relates to a silicone composition comprising a type I photoinitiator and an organopolysiloxane having at least one (meth)acrylate group.

### TECHNICAL FIELD

The subject of the present invention is a type I photoinitiator for the free-radical curing of compositions which are curable by radiation. In particular, the invention relates to a silicone composition comprising a type I photoinitiator and an organopolysiloxane comprising at least one (meth)acrylate group.

### TECHNOLOGICAL BACKGROUND

The use of plastic films as substrate materials for the surface application of silicone coatings in order to create release coatings (non-stick coatings) requires an appropriate technology. Indeed, most of these plastic films are heat-sensitive. A dimensional deformation of the film thus occurs during coating and the drying of the silicone layer in thermal ovens, under the combined effect of the tensile forces and of the temperature to which the films are subjected. The technology of curing functional silicone oils under radiation, in particular under ultraviolet (UV) radiation, makes it possible to eliminate the use of high temperatures and therefore to cure the release coating layers without impacting the substrates. In addition, this technology has the advantage of achieving high productivity without being energy-intensive and without the use of solvents. Plastic substrates are the materials of choice for many applications, and their use is constantly growing.

The preparation of silicone release coatings is generally carried out as follows: a silicone composition is applied to a substrate within an industrial coating device comprising rollers operating at very high speed (for example 600 m/min). Once applied to the substrate, the silicone composition is cured to form a solid silicone (e.g. elastomer) release coating. The coated substrate obtained is also called a silicone liner. This silicone liner can in particular be laminated with an adhesive, because the silicone release coating facilitates the removal of adhesive materials reversibly bonded onto these substrates. These silicone liners can thus be used in the field of self-adhesive labels, strips including envelopes, graphic arts, medical care and health applications.

The silicone compositions used to form release coatings are generally cured (crosslinked) under radiation, in particular under UV or visible radiation emitted by doped or undoped mercury vapor lamps whose emission spectrum extends from 200 nm to 450 nm. Light sources such as light-emitting diodes, better known by the acronym “LED”, which deliver spot UV or visible light can also be used.

The radiation curing of functionalized silicone oils can be done according to two approaches: cationic polymerization of epoxy groups or free-radical polymerization of acrylic functional groups. Free-radical polymerization is inhibited neither by bases nor by humidity. The coating substrates and the additives can thus be more diversified, and interest in such free-radical systems is growing.

Free-radical polymerization under radiation, in particular under UV radiation, of molecules having acrylic functional groups is well documented. From a general point of view, curing under radiation is promoted by a radical photoinitiator molecule. A large amount of literature describes radical photoinitiators and their uses. In the field of free-radical polymerization of acrylic silicone compositions, the photoinitiator molecules commonly used are referred to as type I photoinitiators. Under radiation, these molecules split and produce free radicals. These free radicals induce the polymerization initiation reaction which leads to the hardening of the compositions. Many efforts have been made to have type I photoinitiators possess characteristics which enable their use in silicone-acrylic formulations to obtain release coatings. Throughout the present application, the expression “type I photoinitiators” is understood to mean compounds capable of generating polymerization-initiating free radicals under radiation, by intramolecular homolytic fragmentation.

There are also type II photoinitiator systems comprising a radical photoinitiator and a co-initiator. In type II photoinitiator systems, the photoinitiators used are capable of generating polymerization-initiating free radicals by reaction with another compound called a co-initiator, said reaction causing the transfer of a hydrogen from the co-initiator to said photoinitiator. The photoinitiators used in type II photoinitiator systems are referred to as “type II photoinitiators”.

Type I photoinitiators are commonly used, but they can have disadvantages. In particular, the solubility of these photoinitiators in silicone compositions is not always optimal. In addition, photoinitiators and their breakdown products, such as benzaldehyde, pose health risks and can have an unpleasant odor.

It is therefore necessary to develop type I photoinitiators that can overcome these disadvantages.

In this context, the present invention aims to satisfy at least one of the following objectives.

One of the essential objectives of the invention is to provide a radiation-curable silicone composition comprising a type I photoinitiator, which can be used to form release coatings.

Another essential objective of the invention is to provide a radiation-curable silicone composition comprising a type I photoinitiator, and having improved properties.

Another essential objective of the invention is to provide a radiation-curable silicone composition comprising a type I photoinitiator, and having improved properties in terms of conversion and/or reaction kinetics.

Another essential object of the invention is to provide a radiation-curable silicone composition comprising a type I photoinitiator, where the breakdown products of the photoinitiator possess reduced toxicity and/or a low potential for migrating through coatings.

Another essential objective of the invention is to provide a compound that can be used as a radical photoinitiator in radiation-curable compositions.

Another essential object of the invention is to provide a compound which can be used as a radical photoinitiator and which is soluble in silicone compositions, preferably which is rapidly soluble in silicone compositions.

### BRIEF DESCRIPTION OF THE INVENTION

These objectives, among others, are achieved by the present invention which firstly relates to a radiation-curable silicone composition X comprising:
a. at least one organopolysiloxane A including at least one (meth)acrylate group;
b. at least one radical photoinitiator B, which is a compound of formula (I)

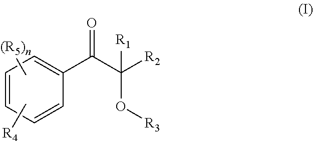

in which
   R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
   R₃ is H or a C₁-C₆ alkyl group, preferably R₃ is H;
   R₄ is a

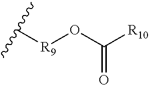

group;
each R₅ group independently represents a C₁-C₆ alkyl group;
n=0, 1, 2, 3, or 4, preferably n=0, 1, or 2;
R₉ is a C₁-C₆ alkylene group or a C₁-C₆ heteroalkylene group; and
R₁₀ is a linear or branched C₁-C₁₈ alkyl group, preferably a linear or branched C₂-C₁₇ alkyl group, more preferably a linear or branched C₄-C₁₃ alkyl group, and even more preferably a linear or branched C₉ alkyl group.

The radical photoinitiator B makes it possible to obtain a silicone composition X having good properties in terms of conversion and reaction kinetics. In addition, the use of the radical photoinitiator B makes it possible to prepare silicone release coatings having good properties. The radical photoinitiator B also allows good curing of the silicone composition X.

Furthermore, the breakdown products from the radical photoinitiator B have a lower migration potential than existing commercial photoinitiators.

The radical photoinitiator B also has good solubility in silicones. It is thus possible to use the pure photoinitiator, diluting it directly in the organopolysiloxane A. Advantageously, the radical photoinitiator B can be dissolved in the organopolysiloxane A in less than 15 hours, or in less than 10 hours, or in less than 5 hours, or in less than 2 hours. For example, the solubility can be determined by adding between 1.5 and 3 parts by weight of radical photoinitiator B into 100 parts by weight of organopolysiloxane A.

Another advantage of the radical photoinitiator B is the transparency of the elastomer obtained after curing the silicone composition X.

The invention also relates to the use of the silicone composition X as described in the present application, for the preparation of a silicone elastomer capable of being used as a release coating on a substrate.

The invention also relates to a silicone elastomer obtained by curing a silicone composition X as described in the present application.

The invention also relates to a method for preparing a coating on a substrate, comprising the following steps:
applying a silicone composition X as described in the present application, and
curing said composition by electron or photon irradiation, preferably by exposure to an electron beam, by exposure to gamma rays, or by exposure to radiation having a wavelength of between 200 nm and 450 nm, in particular to UV radiation.

The invention also relates to a coated substrate that can be obtained by this method.

The invention also relates to the use of the composition X according to the invention, for the preparation of silicone elastomer items by an additive manufacturing process.

The invention also relates to a compound of formula (I)

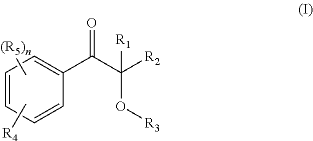

in which
R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
   R₃ is H or a C₁-C₆ alkyl group, preferably R₃ is H;
   R₄ is a

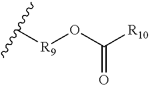

group;
each R₅ group independently represents a C₁-C₆ alkyl group;
n=0, 1, 2, 3, or 4, preferably n=0, 1, or 2;
R₉ is a C₁-C₆ alkylene group or a C₁-C₆ heteroalkylene group; and
R₁₀ is a linear or branched C₂-C₁₈ alkyl group, preferably a linear or branched C₄-C₁₃ alkyl group, and more preferably a linear or branched C₉ alkyl group.

The invention also relates to the use of a compound as defined in the present application, as a radical photoinitiator.

### Definitions

In the present application, the term “radiation-curable silicone composition” is understood to mean a silicone composition comprising at least one organopolysiloxane capable of hardening by electron or photon irradiation. Electron irradiation includes exposure to an electron beam. Photon irradiation includes exposure to radiation having a wavelength of between 200 nm and 450 nm, in particular UV radiation, or exposure to gamma rays.

“(Meth)acrylate” is understood to mean a methacrylate group or an acrylate group.

“Alkyl” is understood to mean a linear or branched alkyl group. The alkyl group preferably comprises 1 to 6 carbon atoms.

“Alkylene” is understood to mean a divalent, linear or branched alkyl group. The alkylene group preferably comprises between 1 and 6 carbon atoms, and more preferably between 1 and 4 carbon atoms.

“Heteroalkylene” is understood to mean a divalent, linear or branched heteroalkyl group. The heteroalkyl group preferably comprises between 1 and 6 carbon atoms, and between 1 and 3 heteroatoms selected from the group consisting of O, N, and S, where N and S can optionally be oxidized. Heteroatoms can be placed at any position in the heteroalkyl group, interior or at one end.

In this application, all percentages are indicated as % by weight, unless stated otherwise.

### DETAILED DESCRIPTION

### Curable Silicone Composition X

The invention firstly relates to a radiation-curable silicone composition X comprising:
a. at least one organopolysiloxane A including at least one (meth)acrylate group;
b. at least one radical photoinitiator B, which is a compound of formula (I)

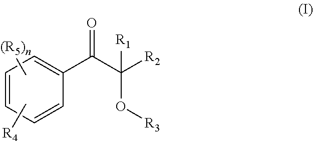

in which
   R₁ and R₂ are chosen, independently of each other, from C₁-C₈ alkyl groups and C₃-C₇ cycloalkyl groups;
or R₁ and R₂, together with the carbon atom to which they are attached, form a C₃-C₇ cycloalkyl group;
   R₃ is H or a C₁-C₆ alkyl group, preferably R₃ is H;
   R₄ is a

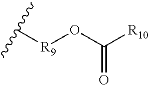

group;
each R₅ group independently represents a C₁-C₆ alkyl group;
n=0, 1, 2, 3, or 4, preferably n=0, 1, or 2;
R₉ is a C₁-C₆ alkylene group or a C₁-C₆ heteroalkylene group; and
R₁₀ is a linear or branched C₁-C₁₈ alkyl group, preferably a linear or branched C₂-C₁₇ alkyl group, more preferably a linear or branched C₄-C₁₃ alkyl group, and even more preferably a linear or branched C₉ alkyl group.

According to one embodiment, the silicone composition X is curable by photon irradiation, preferably by exposure to radiation having a wavelength of between 200 nm and 450 nm, in particular to UV radiation.

According to one embodiment, the radiation-curable silicone composition X has a viscosity of between 50 and 2500 mPa·s, preferably between 100 and 1500 mPa·s. It is therefore possible to use it with the coating tools used for preparing silicone release coatings.

All the viscosities referred to in this description correspond to a magnitude of dynamic viscosity at 25° C., meaning the dynamic viscosity which is measured, in a manner known per se, using a Brookfield viscometer at a sufficiently low shear rate gradient for the measured viscosity to be independent of the rate gradient.

### Organopolysiloxane A

According to the invention, the curable silicone compositions X according to the invention comprise at least one organopolysiloxane A including at least one (meth)acrylate group, preferably at least two (meth)acrylate groups.

As representative of the (meth)acrylate functional groups carried by the silicone and most particularly suitable for the invention, particular mention can be made of the derivatives of acrylates, methacrylates, ethers of (meth)acrylates and esters of (meth)acrylates linked to the polysiloxane chain by an Si—C bond.

According to one embodiment, the organopolysiloxane A comprises:
a) at least one unit having the following formula (IV):

RₐZ_{b}SiO_{(4-a-b)/2} (IV)

a formula in which:
the R symbols, which are identical or different, each represent a linear or branched C₁ to C₁₈ alkyl group, a C₆ to C₁₂ aryl or aralkyl group, said alkyl and aryl groups possibly being substituted, preferably by halogen atoms, or an —OR⁵ group with R₅ being a hydrogen atom or a hydrocarbon group comprising from 1 to 10 carbon atoms,
the Z symbols are monovalent groups of formula -y-(Y′)n in which:
y represents a polyvalent C₁-C₁₈ alkylene or heteroalkylene group, said alkylene and heteroalkylene groups able to be linear or branched, and possibly being interrupted by one or more cycloalkylene groups, and possibly being extended by bivalent oxyalkylene or polyoxyalkylene radicals at C₁ to C₄, said alkylene, heteroalkylene, oxyalkylene and polyoxyalkylene groups possibly being substituted by one or more hydroxy groups,
   Y′ represents a monovalent alkenylcarbonyloxy group, and
   n is equal to 1, 2, or 3, and
   a is an integer equal to 0, 1, or 2, b is an integer equal to 1 or 2, and the sum a+b=1, 2, or 3; and
b) optionally, units having the following formula (V):

RₐSiO_{(4-B)2} (V)
a formula in which:
   the R symbols are as defined above in formula (IV), and
   a is an integer equal to 0, 1, 2, or 3.

In formulas (IV) and (V) above, the R symbols, which are identical or different, each represent a linear or branched C₁ to C₁₈ alkyl group or a C₆ to C₁₂ aryl or aralkyl group. Preferably, the R symbol represents a monovalent group chosen from the group composed of methyl, ethyl, propyl, 3,3,3-trifluoropropyl, xylyl, tolyl, and phenyl, and more preferably the R symbol represents a methyl.

The organopolysiloxane A can have a linear, branched, cyclic or network structure. Preferably, the organopolysiloxane A has a linear structure. When linear organopolysiloxanes are concerned, these can essentially consist of:
“D” siloxyl units chosen among units of formulas R₂SiO_{2/2}, R₂SiO_{2/2}, and Z₂SiO_{2/2};
“M” siloxyl units chosen among units of formulas R₃SiO_{1/2}, R₂ZSiO_{1/2}, RZ₂SiO_{1/2}, and Z₃SiO_{1/2}, and
the R and Z symbols are as defined above in formula (I).

According to one embodiment, in formula (IV) above, the aforementioned Y′ alkenylcarbonyloxy groups include acryloxy [CH₂═CH—CO—O—] and the methacryloxy group: [CH₂═C(CH₃)—CO—O—]. Advantageously, the organopolysiloxane A comprises at least two Y′ alkenylcarbonyloxy groups, preferably at least three Y′ alkenylcarbonyloxy groups.

As illustrations of the y symbol in the units of formula (IV), the following groups can be mentioned:
—CH₂—;
—(CH₂)₂—;
—(CH₂)₃—;
—CH₂—CH(CH₃)—CH₂—;
—(CH₂)₃—NR′—CH₂—CH₂—; where R′ is a C₁-C₆ alkyl group
—(CH₂)₃—OCH₂—;
—(CH₂)₃—[O—CH₂—CH(CH₃)—]ₙ—; where n=1 to 25
—(CH₂)₃—O—CH₂—CH(OH)(—CH₂—);
—(CH₂)₃—O—CH₂—C(CH₂—CH₃)[—(CH₂—)]₂;
—(CH₂)₃—O—CH₂—C[—(CH₂)—]₃; and
—(CH₂)₂—C₆H₉(OH)—.

Preferably, organopolysiloxane A corresponds to the following formula (VI):

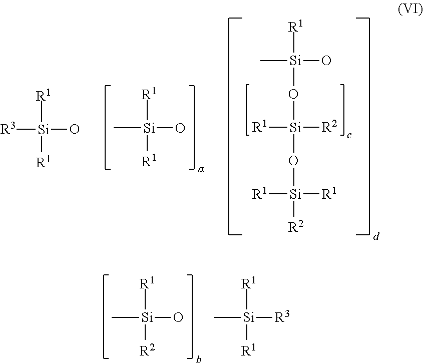

a formula in which:
the R¹ symbols, which are identical or different, each represent a linear or branched C₁ to C₁₈ alkyl group, a C₆ to C₁₂ aryl or aralkyl group, said alkyl and aryl groups possibly being substituted, preferably by halogen atoms, or an —OR⁵ group with R⁵ being a hydrogen atom or a hydrocarbon group comprising from 1 to 10 carbon atoms,
the R² and R³ symbols, which are identical or different, each represent either an R¹ group or a monovalent group of formula Z=-y-(Y′)n in which:
y represents a polyvalent C₁-C₁₈ alkylene or heteroalkylene group, said alkylene and heteroalkylene groups able to be linear or branched, and possibly being interrupted by one or more cycloalkylene groups, and possibly being extended by bivalent oxyalkylene or polyoxyalkylene radicals at C₁ to C₄, said alkylene, heteroalkylene, oxyalkylene, and polyoxyalkylene groups possibly being substituted by one or more hydroxy groups,
Y′ represents a monovalent alkenylcarbonyloxy group,
n is equal to 1, 2, or 3, and
with a=0 to 1000, b=0 to 500, c=0 to 500, d=0 to 500, and a+b+c+d=0 to 2500, preferably a=0 to 500 and a+b+c+d=0 to 500,
on condition that at least one R² or R³ symbol represents the monovalent group of formula Z, preferably at least two R² or R³ symbols represent a monovalent group of formula Z.

According to a preferred embodiment, in formula (VI) above:
c=0, d=0, a=1 to 1000, b=1 to 250, the symbol R² represents the monovalent group of formula Z, and the symbols R¹ and R³ have the same meaning as above.

Even more preferably, in formula (VI) above:
c=0, d=0, a=1 to 500, b=2 to 100, the symbol R² represents the monovalent group of formula Z, and the symbols R¹ and R³ have the same meaning as above.

According to one embodiment, the organopolysiloxane A according to the invention corresponds to one of the following formulas (VII), (VIII), (IX), or (X):

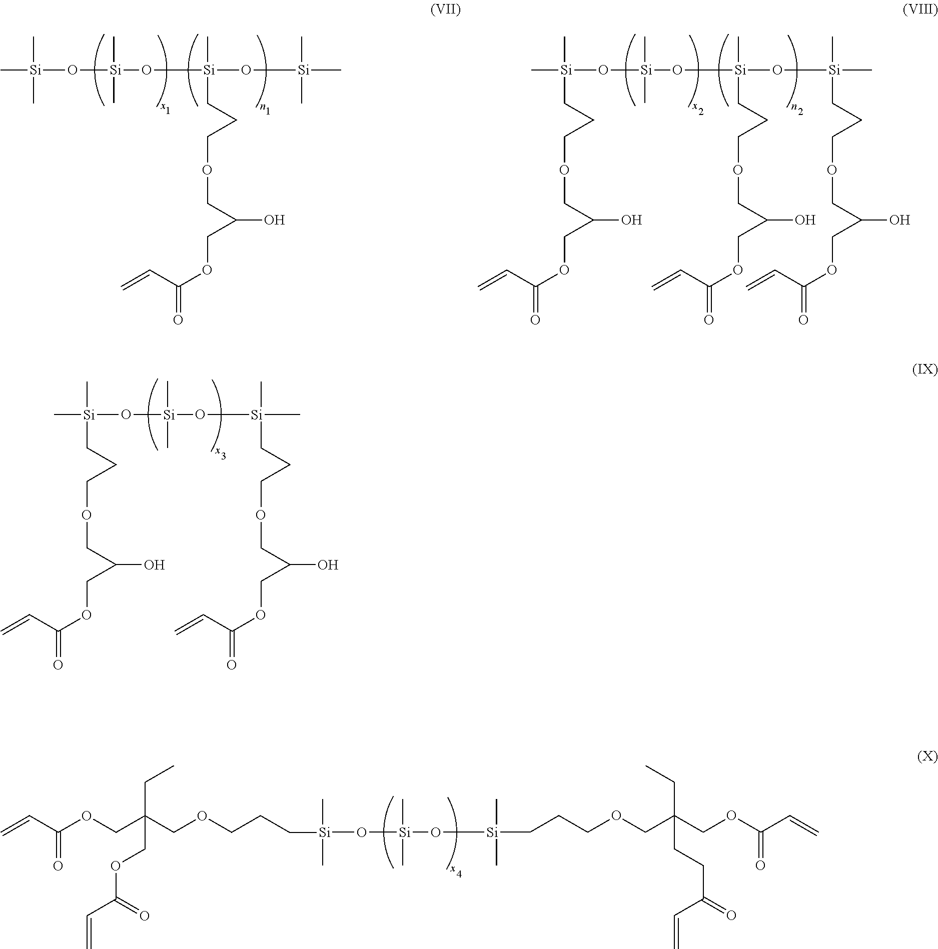

where:
x1 is between 1 and 1000; preferably x1 is between 1 and 500,
n1 is between 1 and 100, preferably n1 is between 2 and 100,
x2 is between 1 and 1000, preferably x2 is between 1 and 500
n2 is between 1 and 100, preferably n2 is between 2 and 100,
x3 is between 1 and 1000, preferably x3 is between 1 and 500, and
x4 is between 1 and 1000, preferably x4 is between 1 and 500.

The radiation-curable silicone composition X can comprise between 25 and 99.99% by weight of organopolysiloxane A, relative to the total weight of the radiation-curable silicone composition X. Preferably, the radiation-curable silicone composition X can comprise between 50 and 99.5% of organopolysiloxane A, relative to the total weight of the radiation-curable silicone composition X.

Of course, depending on the variants, the organopolysiloxane A can be a mixture of compounds satisfying the definition of organopolysiloxane A.

### Radical Photoinitiator B

The radical photoinitiator B is a type I photoinitiator. After photon irradiation, the radical photoinitiator B undergoes homolytic cleavage at the a position of the carbonyl functional group with the formation of two radical fragments, one of which is a benzoyl radical substituted by an R₄ group.

The photoinitiator B allows improving the properties of the silicone composition X, in particular in terms of conversion and reaction kinetics. In addition, the radical photoinitiator B makes it possible to obtain good curing of the silicone composition X.

The silicone composition X can comprise between 0.01 and 20% by weight of the radical photoinitiator B, relative to the total weight of the radiation-curable silicone composition X. Preferably, the radiation-curable silicone composition X comprises between 0.1 and 10% by weight of the radical photoinitiator B, and preferably between 0.1% and 5% by weight.

The radical photoinitiator B is a compound of formula (I)

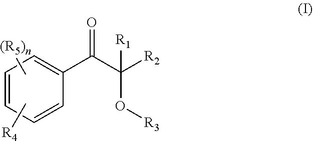

in which
R₁ and R₂ are chosen, independently of each other, from C₁-C₆ alkyl groups and C₃-C₇ cycloalkyl groups;
   or R₁ and R₂, together with the carbon atom to which they are attached, form a C₃-C₇ cycloalkyl group;
R₃ is H or a C₁-C₆ alkyl group, preferably R₃ is H;
R₄ is a

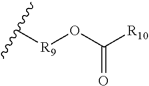

group;
each R₅ group independently represents a C₁-C₈ alkyl group;
n=0, 1, 2, 3, or 4, preferably n=0, 1, or 2;
R₉ is a C₁-C₆ alkylene group or a C₁-C₆ heteroalkylene group; and
R₁₀ is a linear or branched C₁-C₁₈ alkyl group, preferably a linear or branched C₂-C₁₇ alkyl group, more preferably a linear or branched C₄-C₁₃ alkyl group, and even more preferably a linear or branched C₉ alkyl group.

According to one embodiment, the compound of formula (I) is a compound of formula (II)

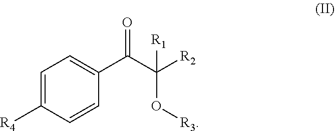

According to one embodiment, the compound of formula (I) is a compound of formula (III)

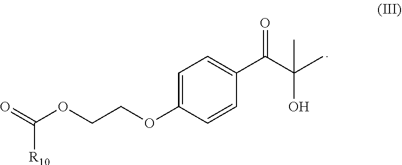

Advantageously, R₁ and R₂ are chosen, independently of each other, from C₁-C₆ alkyl groups. Preferably, R₁ and R₂ are each a methyl group.

Advantageously, R₃ is H.

According to one embodiment, n=0. According to another embodiment, n=1 or 2, and each R₅ group independently represents a C₁-C₆ alkyl group, preferably a methyl group.

According to one embodiment, R₉ is a C₁-C₆ heteroalkylene group, in particular a —O—(CH₂)₂— group, the oxygen atom being attached to the phenyl.

R₁₀ is a linear or branched C₁-C₁₈ alkyl group, preferably a linear or branched C₂-C₁₇ or C₂-C₁₈ alkyl group, more preferably a linear or branched C₄-C₁₃ or C₃-C₁₀ alkyl group, and even more preferably a linear or branched C₉ alkyl group.

According to one embodiment, R₁₀ is a linear or branched C₁-C₁₈ alkyl group, preferably a linear or branched C₁-C₁₃ alkyl group, more preferably a linear or branched C₁-C₁₀ alkyl group, and even more preferably a linear or branched C₁-C₉ alkyl group.

According to one embodiment, R₁₀ is a linear or branched C₂-C₁₈ alkyl group, preferably a linear or branched C₂-C₁₃ alkyl group, more preferably a linear or branched C₂-C₁₀ alkyl group, and even more preferably a linear or branched C₂-C₉ alkyl group.

According to one embodiment, R₁₀ is a branched C₁-C₁₈ alkyl group, preferably a branched C₂-C₁₇ or C₃-C₁₈ alkyl group, more preferably a branched C₄-C₁₃ alkyl group, and even more preferably a branched C₉ alkyl group.

Examples of an R₁₀ group include the branched C₄ alkyl group, branched C₆ alkyl group, branched C₈ alkyl group, branched C₉ alkyl group, branched C₁₁ alkyl group, and branched C₁₃ alkyl group.

When R₁₀ is a branched alkyl group, it can include a quaternary carbon. Preferably, the quaternary carbon is in the carbonyl's alpha position: the term trialkyl acetic acid esters is then used. The trialkyl acetic acids can come from cut oil. According to one embodiment, the R₁₀—(CO)—O— group represents a trialkyl acetic acid ester, and, preferably, R₁₀ represents a branched C₄, C₆, C₈, C₉, C₁₁, or C₁₃ alkyl group.

In some cases, when the R₁₀—(CO)—O— represents a trialkyl acetic acid ester derived from cut oil, several constitutional isomers may be present. In particular, this may be the case when R₁₀ is a branched C₆, C₈, C₉, C₁₁, or C₁₃ alkyl group. Thus, R₁₀ can represent a mixture of constitutional isomers. For example, when R₁₀ represents a branched C₉ alkyl group, this alkyl group can comprise different isomers of the following types: —C(CH₃)₂—CH(CH₃)—CH₂—CH(CH₃)₂, —C(CH₃)(CH(CH₃)₂)—CH₂—CH(CH₃)₂, —C(CH₃)₂—(CH₂)₅—CH₃, and —C(CH₂—CH₃)₂—(CH₂)₃—CH₃.

### Other Additives

The radiation-curable silicone composition X may also comprise other additives such as polymerization inhibitors, fillers, virucides, bactericides, anti-abrasion additives, and pigments (organic or inorganic).

For the polymerization inhibitors, mention can be made of phenols, hydroquinone, 4-OMe-phenol, 2,4,6-tri-tert-butylphenol (BHT), phenothiazine, and nitroxyl radicals such as (2,2,6,6-tetramethylpiperidin-1-yl)oxyl (TEMPO).

The radiation-curable silicone composition X may also comprise an organic compound C comprising at least one (meth)acrylate functional group. Organic compound C comprising at least one (meth)acrylate functional group is understood to mean any compound comprising one or more (meth)acrylate functional groups. According to one embodiment, the organic compound C comprising at least one (meth)acrylate functional group does not comprise a siloxane structure.

Suitable in particular as organic compounds C comprising a (meth)acrylate functional group, are epoxy (meth)acrylates, polyesters of glyceryl (meth)acrylate, urethane (meth)acrylates, polyethers of (meth)acrylate, polyester (meth)acrylates, and (meth)acrylate acrylics. More particularly preferred are trimethylolpropane triacrylate, tripropylene glycol diacrylate, hexanediol diacrylate, and pentaerythritol tetraacrylate.

Examples of organic compound C comprising a (meth)acrylate functional group include: ethylhexyl acrylate, stearyl acrylate, tetrahydrofurfuryl acrylate, lauryl acrylate, isodecyl acrylate, 2(2-ethoxyethoxy)ethyl acrylate, cyclohexyl acrylate, isooctyl acrylate, tridecyl acrylate, isobornyl acrylate, caprolactone acrylate, alkoxylated phenol acrylates, 1,3-butylene glycol diacrylate, 1,4-butanediol diacrylate, diethylene glycol diacrylate, neopentyl glycol diacrylate, tetraethylene glycol diacrylate, triethylene glycol diacrylate, dipropylene glycol diacrylate, alkoxylated hexanediol diacrylates, trimethylolpropane triacrylate, ethoxylated trimethylolpropane triacrylate, propoxylated glycerol triacrylate, pentarythritol triacrylate, pentaerythritol tetraacrylate, di-trimethylolpropane tetraacrylate, and dipentaerythritol pentaacrylate.

The radiation-curable silicone composition X may also comprise a filler. The radiation-curable silicone composition X can comprise between 0.1 and 40% by weight of filler, relative to the total weight of the radiation-curable silicone composition X. According to one embodiment, the radiation-curable silicone composition X comprises between 20 to 30% by weight of filler. According to another embodiment, the radiation-curable silicone composition X comprises between 0.1 and 10% by weight of filler. This filler is preferably inorganic. The filler can be a very finely divided product with an average particle diameter of less than 0.1 μm. The filler can in particular be siliceous. With regard to siliceous materials, they can act as a reinforcing or semi-reinforcing filler. Reinforcing siliceous fillers are chosen from colloidal silicas, combustion and precipitated silica powders, or mixtures thereof. These powders have an average particle size that is generally less than 0.1 μm (micrometers), and a BET specific surface area greater than 30 m²/g, preferably between 30 and 350 m²/g. Semi-reinforcing siliceous fillers such as diatomaceous earth or crushed quartz can also be used. These silicas can be incorporated as is or after having been treated with organosilicon compounds conventionally used for this purpose. Among these compounds are: methylpolysiloxanes such as hexamethyldisiloxane, octamethylcyclotetrasiloxane; methylpolysilazanes such as hexamethyldisilazane, hexamethylcyclotrisilazane, tetramethyldivinyldisilazane; chlorosilanes such as dimethyldichlorosilane, trimethylchlorosilane, methylvinyldichlorosilane, dimethylvinylchlorosilane; alkoxysilanes such as dimethyldimethoxysilane, dimethylvinyl methoxysilane, trimethyl methoxysilane, and mixtures thereof. With regards to the non-siliceous inorganic materials, they can act as a semi-reinforcing or packing inorganic filler. Examples of these non-siliceous fillers which can be used alone or as a mixture are calcium carbonate, possibly surface-treated with an organic acid or with an ester of an organic acid, calcined clay, titanium oxide in rutile form, oxides of iron, zinc, chromium, zirconium, or magnesium, the various forms of alumina (hydrated or not), boron nitride, lithopone, barium metaborate, barium sulfate, and glass microspheres. These fillers are more coarse, typically having an average particle diameter greater than 0.1 μm and a specific surface area that is generally less than 30 m²/g. These fillers may have been surface-modified by treatment with the various organosilicon compounds usually employed for this purpose.

According to one embodiment, the radiation-curable silicone composition X comprises:
a. between 25 and 99.99% by weight of at least one organopolysiloxane A comprising at least one (meth)acrylate group;
b. between 0.01 and 20% by weight of at least one radical photoinitiator B, which is a compound of formula (I)

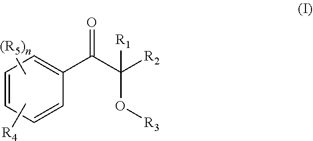

in which
   R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
   R₃ is H or a C₁-C₆ alkyl group, preferably R₃ is H;
   R₄ is a

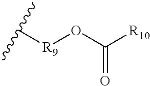

group;
each R₅ group independently represents a C₁-C₆ alkyl group;
n=0, 1, 2, 3, or 4, preferably n=0, 1 or 2;
R₉ is a C₁-C₆ alkylene group or a C₁-C₆ heteroalkylene group; and
R₁₀ is a linear or branched C₁-C₁₈ alkyl group, preferably a linear or branched C₂-C₁₇ alkyl group, more preferably a linear or branched C₄-C₁₃ alkyl group, and even more preferably a linear or branched C₉ alkyl group.

### Applications

The invention also relates to the use of the radiation-curable silicone composition X for the preparation of silicone elastomers. These silicone elastomers can have release properties relative to adhesives.

The invention also relates to a method for preparing a silicone elastomer, comprising a step of curing a radiation-curable silicone composition X.

According to one embodiment of the method of the invention, the curing step is carried out in air or in an inert atmosphere. Preferably, this curing step is carried out in an inert atmosphere.

According to one embodiment, the curing step of the method according to the invention is carried out by UV radiation having a wavelength of between 200 nm and 450 nm, preferably in an inert atmosphere.

According to another embodiment, the curing step of the method according to the invention is carried out by exposure to an electron beam or to gamma rays.

The UV radiation can be emitted by doped or undoped mercury vapor lamps whose emission spectrum extends from 200 nm to 450 nm. Light sources such as light-emitting diodes, better known by the acronym “LED”, which deliver spot UV or visible light can also be used.

According to a preferred embodiment of the invention, the radiation is ultraviolet light of a wavelength of less than 400 nanometers. According to a preferred embodiment of the invention, the radiation is ultraviolet light of a wavelength greater than 200 nanometers.

According to one advantageous embodiment, LED UV lamps are used (UV emissions at 365, 375, 385, and/or 395 nm).

A dose of ultraviolet radiation within the range of about 0.1 to about 0.5 joules is generally sufficient to induce crosslinking.

The irradiation time can be short and is generally less than 1 second and is on the order of a few hundredths of a second for low coating thicknesses. The curing obtained is excellent even in the absence of any heating.

According to one embodiment, the curing step is carried out at a temperature between 10° C. and 50° C., preferably between 15° C. and 35° C.

Of course, the curing speed can be adjusted in particular by the number of UV lamps used, by the duration of the exposure to UV, and by the distance between the composition and the UV lamp.

The invention also relates to a method for preparing a coating on a substrate, comprising the following steps:
applying a radiation-curable silicone composition X onto a substrate, and
curing said composition by electron or photon irradiation, preferably by exposure to an electron beam, by exposure to gamma rays, or by exposure to radiation having a wavelength of between 200 nm and 450 nm, in particular UV radiation.

The solventless composition X according to the invention, i.e. undiluted, can be applied using devices capable of uniformly depositing small quantities of liquids. For this purpose, one can use for example the device known as the “Helio glissant” comprising in particular two superimposed rollers: the role of the lower roller, which is immersed into the coating tank where the compositions are located, is to impregnate the upper roller in one very thin layer, the role of the latter then being to deposit on the paper the desired amounts of the compositions with which it is impregnated, such dosing being obtained by adjusting the respective speed of the two rollers which rotate in opposite directions relative to each other.

Curing, which results in a hardening of the silicone composition X, can be carried out continuously by passing the substrate coated with the composition through an irradiation device which is designed to ensure sufficient dwell time for the coated substrate to complete the curing of the coating. Preferably, the curing is carried out in the presence of the lowest possible concentration of oxygen, typically at an oxygen concentration of less than 100 ppm, and preferably less than 50 ppm. The curing is generally carried out in an inert atmosphere, for example nitrogen or argon. The exposure time required to cure the silicone composition X varies with factors such as:
the particular formulation used, the radiation type and wavelength,
the dose flow rate, the energy flux,
the concentration of radical photoinitiator, and
the atmosphere and the thickness of the coating.

These parameters are well known to those skilled in the art, who will know how to adapt them.

The amounts of composition X deposited on the substrates are variable and most often range between 0.1 and 5 g/m² of treated surface. These amounts depend on the nature of the substrates and on the release properties desired. They are most often between 0.5 and 1.5 g/m² for non-porous substrates.

This method is particularly suitable for preparing a silicone release coating on a substrate which is a flexible substrate made of textile, paper, polyvinyl chloride, polyester, polypropylene, polyamide, polyethylene, polyethylene terephthalate, polyurethane, or non-woven fiberglass.

Flexible substrates coated with a silicone release coating can be for example:
a paper or a polymer film of the polyolefin type (polyvinyl chloride (PVC), PolyPropylene or Polyethylene) or of the polyester type (PolyEthylene Terephthalate or PET),
an adhesive tape whose inner face is coated with a layer of pressure-sensitive adhesive and whose outer face comprises the silicone release coating;
or a polymer film for protecting the adhesive face of a self-adhesive or pressure-sensitive adhesive element.

These coatings are particularly suitable for their use in the release coating field.

The invention also relates to a coated substrate that can be obtained according to the method described above. As indicated above, the substrate can be a flexible substrate made of textile, paper, polyvinyl chloride, polyester, polypropylene, polyamide, polyethylene, polyethylene terephthalate, polyurethane, or non-woven glass fibers.

The coated substrates have non-stick and water-repellent features, or allow improved surface properties such as slipperiness, stain resistance, or softness.

Another object of the invention relates to the use of a substrate at least partially coated with a release coating according to the invention and as defined above, in the field of self-adhesive labels, strips including envelopes, graphic arts, medical care and health applications.

The invention also relates to the use of the composition X according to the invention, for the preparation of silicone elastomer items by an additive manufacturing process. Additive manufacturing processes are also known as 3D printing processes. This description in general includes the ASTM F2792-12a designation, “Standard Terminology for Additive Manufacturing Technologies.” According to this ASTM standard, a “3D printer” is defined as “a machine used for 3D printing”, and “3D printing” is defined as “the fabrication of objects through the deposition of a material using a printhead, nozzle, or another printer technology”.

Additive manufacturing “AM” is defined as a process of joining materials in order to manufacture objects from 3D model data, usually layer upon layer, as opposed to subtractive manufacturing processes. Synonyms associated with 3D printing and encompassed by 3D printing include additive fabrication, additive processes, additive techniques, and layer manufacturing. Additive manufacturing (AM) can also be called rapid prototyping (RP). As used herein, “3D printing” is interchangeable with “additive manufacturing” and vice versa.

Irradiation of the layers of the silicone compositions X as printing progresses allows rapid gelation of at least part of the composition during production, and thus each layer retains its shape without the printed structure collapsing.

Advantageously, the silicone compositions X according to the invention can be used for 3D printing processes which make use of vat photopolymerization (Digital Light Processing, stereolithography), material extrusion, deposition of materials, or inkjet, adapting the viscosity of the silicone composition X to the technology employed.

### Compound of Formula (I)

The invention also relates to a compound of formula (I)

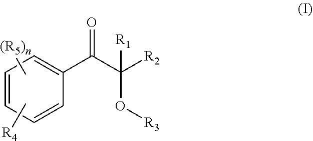

in which
R₁ and R₂ are chosen, independently of each other, from C₁-C₆ alkyl groups and C₃-C₇ cycloalkyl groups;
   or R₁ and R₂, together with the carbon atom to which they are attached, form a C₃-C₇ cycloalkyl group;
R₃ is H or a C₁-C₆ alkyl group, preferably R₃ is H;
R₄ is a

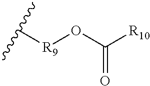

group;
each R₅ group independently represents a C₁-C₆ alkyl group;
n=0, 1, 2, 3, or 4, preferably n=0, 1, or 2;
R₉ is a C₁-C₆ alkylene group or a C₁-C₆ heteroalkylene group; and
R₁₀ is a linear or branched C₁-C₁₈ alkyl group, preferably a linear or branched C₂-C₁₇ or C₂-C₁₈ alkyl group, more preferably a linear or branched C₄-C₁₃ alkyl group, and even more preferably a linear or branched C₉ alkyl group.

According to one embodiment, the compound of formula (I) is a compound of formula (II)

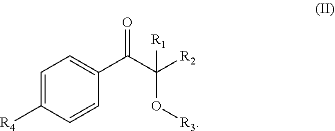

According to one embodiment, the compound of formula (I) is a compound of formula (III)

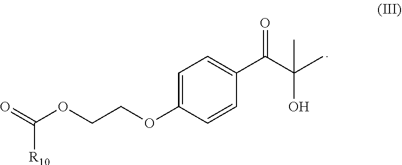

Advantageously, R₁ and R₂ are chosen, independently of each other, from C₁-C₆ alkyl groups. Preferably, R₁ and R₂ are each a methyl group.

### Advantageously, R₃ is H.

According to one embodiment, n=0. According to another embodiment, n=1 or 2, and each R₅ group independently represents a C₁-C₆ alkyl group, preferably a methyl group.

According to one embodiment, R₉ is a C₁-C₆ heteroalkylene group, in particular a —O—(CH₂)₂— group, the oxygen atom being attached to the phenyl.

R₁₀ is a linear or branched C₁-C₁₈ alkyl group, preferably a linear or branched C₂-C₁₇ or C₂-C₁₈ alkyl group, more preferably a linear or branched C₄-C₁₃ or C₃-C₁₀ alkyl group, and even more preferably a linear or branched C₉ alkyl group.

According to one embodiment, R₁₀ is a linear or branched C₁-C₁₈ alkyl group, preferably a linear or branched C₁-C₁₃ alkyl group, more preferably a linear or branched C₁-C₁₀ alkyl group, and even more preferably a linear or branched C₁-C₉ alkyl group.

According to one embodiment, R₁₀ is a linear or branched C₂-C₁₈ alkyl group, preferably a linear or branched C₂-C₁₃ alkyl group, more preferably a linear or branched C₂-C₁₀ alkyl group, and even more preferably a linear or branched C₂-C₉ alkyl group.

According to one embodiment, R₁₀ is a branched C₁-C₁₈ alkyl group, preferably a branched C₂-C₁₇ or C₃-C₁₈ alkyl group, more preferably a branched C₄-C₁₃ alkyl group, and even more preferably a branched C₉ alkyl group.

Examples of an R₁₀ group include the branched Ca alkyl group, branched C₆ alkyl group, branched C₈ alkyl group, branched C₉ alkyl group, branched C₁₁ alkyl group, and branched C₁₃ alkyl group.

When R₁₀ is a branched alkyl group, it can include a quaternary carbon. Preferably, the quaternary carbon is in the carbonyl's alpha position: the term trialkyl acetic acid esters is then used. The trialkyl acetic acids can come from cut oil. According to one embodiment, the R₁₀—(CO)—O— group represents a trialkyl acetic acid ester, and, preferably, R₁₀ represents a branched C₄, C₆, C₈, C₉, C₁₁, or C₁₃ alkyl group.

In some cases, when the R₁₀—(CO)—O— group represents a trialkyl acetic acid ester derived from cut oil, several constitutional isomers may be present. In particular, this may be the case when R₁₀ is a branched C₆, C₈, C₉, C₁₁, or C₁₃ alkyl group. Thus, R₁₀ can represent a mixture of constitutional isomers. For example, when R₁₀ represents a branched C₉ alkyl group, this alkyl group can comprise different isomers of the following types: —C(CH₃)₂—CH(CH₃)—CH₂—CH(CH₃)₂, —C(CH₃)(CH(CH₃)₂)—CH₂—CH(CH₃)₂, —C(CH₃)₂—(CH₂)₅—CH₃, and —C(CH₂—CH₃)₂—(CH₂)₃—CH₃.

The compounds of formula (I) can be synthesized according to the standard methods used in organic chemistry, known to those skilled in the art.

In particular, the compounds of formula (III) can be synthesized from a compound of formula (XI) or from a compound of formula (XII) according to the conventional methods used in organic chemistry, known to those skilled in the art.

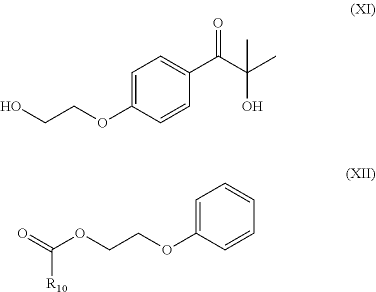

Many access routes are possible, such as:
direct esterification of the corresponding acid R₁₀—COOH by the compound of formula (XI) in the presence of a strong acid and a solvent enabling distillation of its azeotrope with the water formed,
transesterification of a methyl or ethyl ester of the corresponding acid R₁₀—COO— by the compound of formula (XI), catalyzed for example by a beta-diketonate of a group IV metal, in particular zirconium tetra-acetylacetonate,
the reaction of compound (XI) with the chloride of the corresponding acid R₁₀—COCl in the presence of triethylamine,
preparation of the compound of formula (XII) from 2-phenoxyethanol, for example by esterification or transesterification, then Friedel-Crafts reaction with isobutyryl chloride, and chlorination or bromination of the ketone obtained, lastly followed by base hydrolysis to form the compound of formula (I).

### Use of the Compound of Formula (I)

The invention also relates to the use of a compound of formula (I) as described above as a radical photoinitiator, in particular as a radical photoinitiator for acrylic silicone compositions.

Indeed, the compound of formula (I) according to the invention is soluble in silicones; it is therefore possible to use it as a radical photoinitiator in these compositions, without adding solvent.

According to another embodiment, it is also possible to use a small amount of solvent to help solubilize the compound of formula (I) in the silicone compositions.

The invention also relates to the use of a compound of formula (I) as described above as a radical photoinitiator in a radiation-curable composition Y comprising at least one radiation-curable unsaturated compound D.

The invention also relates to a radiation-curable composition Y comprising:
at least one radiation-curable unsaturated compound D,
a photoinitiator which is a compound of formula (I) as described above.

The radiation-curable unsaturated compound D may comprise one or more double bonds that are not part of an aromatic ring.

According to one embodiment, the radiation-curable unsaturated compound D is a hydrocarbon compound comprising one or more double bonds, and, optionally, one or more heteroatoms chosen among N, P, O, S, and F. The unsaturated compound D can for example be chosen from the (meth)acrylic acids, (meth)acrylic acid esters, (meth)acrylamides, N-substituted (meth)acrylamides, unsaturated acid anhydrides, styrenes, alkylstyrenes, divinylbenzenes, vinyl ethers, vinyl and allyl esters, isocyanurates, N-vinyl heterocycles, and mixtures thereof.

The unsaturated compound D can be monomeric or oligomeric. When the unsaturated compound D is monomeric, it can comprise from 2 to 40 carbon atoms, and optionally from 1 to 20 heteroatoms, chosen among N, P, O, S, and F. As examples of unsaturated oligomeric compounds D, mention can be made of polymers comprising double bonds in the main chain or in a pendent chain. Among these polymers, mention can be made of unsaturated polyesters, unsaturated polyamides, and unsaturated polyurethanes.

According to another embodiment, the radiation-curable unsaturated compound D is an organopolysiloxane comprising one or more double bonds. Preferably, the radiation-curable unsaturated compound D is an organopolysiloxane comprising at least one (meth)acrylate group. The unsaturated compound D can be an organopolysiloxane A as described above.

The invention also relates to the use of a compound of formula (I) as described above, as a radical photoinitiator in a radiation-curable composition Y comprising at least one radiation-curable unsaturated compound D, said unsaturated compound D being an organopolysiloxane comprising one or more double bonds, preferably an organopolysiloxane comprising at least one (meth)acrylate group.

The radiation-curable composition Y can be used in a wide variety of technical fields such as printing inks, printing techniques, varnishes, coatings for wood, coatings for plastics, coatings for metals, adhesives, and 3D printing.

### EXAMPLES

In the examples below, various organopolysiloxanes A and a type I radical photoinitiator B were used to prepare radiation-curable silicone compositions X according to the invention. Their structures are shown in the tables below. Unless otherwise stated, throughout this document the percentages are expressed as % by weight.

### Organopolysiloxanes A

TABLE 1

| | | acrylate |
|---|---|---|
| | | content |
| | | (mmol/ |
| | | 100 g |
| | | organo- |
| Com- | | polysi- |
| pound | Formula | loxane) |
| A1 | 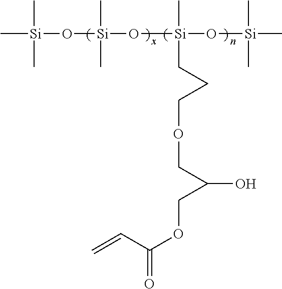 | 90 |
| A2 | 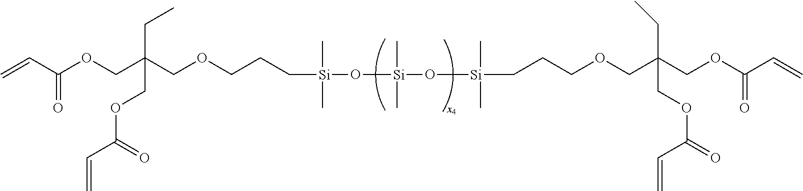 | 53 |
| A3 | 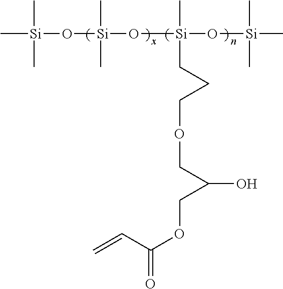 | 200. |

Type I radical photoinitiators B of the following formula:

### B1:

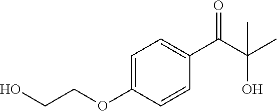 [SMILES: CC(C)(O)C(=O)c1ccc(OCCO)cc1]

commercially available compound, CAS no: 106797-53-9, commercial reference=I2959.

### B2:

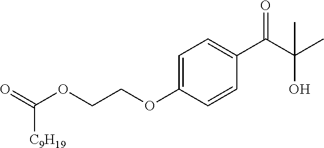

compound according to the invention where CO—C₉H₁₉ represents a group derived from neodecanoic acid.

### B3:

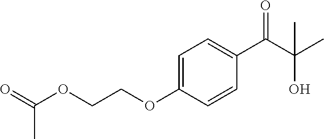 [SMILES: CC(=O)OCCOc1ccc(C(=O)C(C)(C)O)cc1]

This compound was prepared by esterification of compound B1 with acetic acid in the presence of a dehydrating agent. Compound B3 is in the form of a recrystallized solid.

### Example 1: Synthesis of Photoinitiator B2 Et Solubility Study of Compounds B2 and B3 in Silicone Compositions

In a single-neck flask, introduce 1 equivalent of B1, 1 equivalent of neodecanoic acid, and 1 mL of concentrated sulfuric acid per mmol of product. Leave to stir for 2 hours at room temperature under argon. Then leave to react at 120° C. under argon for 12 hours.

Once the reaction is complete, 10 times the volume of the reaction medium is added in water and the mixture is extracted 3 times with n-hexane. The organic phases are then combined, neutralized with sodium carbonate, dried and evaporated. The crude product obtained is then purified on silica gel with a 90/10 cyclohexane/ethyl acetate eluent, to obtain product B2.

Compound B2 was characterized by infrared and NMR. The results are shown in Table 2 below.

### [Table 2]

TABLE 2

| | Instrument/ | |
|---|---|---|
| Analysis | Conditions | Results |
| IR Spectrometry | Shimadzu | Compatible with the expected structure |
| | IRAffinity-1S, | 3479 cm⁻¹: O—H (alcohol) |
| | neat | 2962 cm⁻¹: C—H (alkyl) |
| | | 1728 and 166 cm⁻¹: C═O (ketone and |
| | | ester) |
| | | 1597, 1504, 1458 cm⁻¹: C═C (benzene) |
| | | 1249 and 1149 cm⁻¹: C—O (ether and |
| | | ester) |
| ¹H NMR | Bruker 400 | Compatible with the expected |
| | MHz Solvent: | structure (mixture of isomers) |
| | DMSO-d6 | ¹H signals ppm: 0.56-1.9 (19H, m), |
| | at 25° C. | 1.38 (6H, s), 4.26 (2H, d), 4.40 (2H, |
| | | d), 5.66 (1H, s), 6.99 (2H, d), 8.21 |
| | | (2H, d) |

The solubility of compounds B2 and B3 in silicone compositions was also tested. The results are presented in Table 3 below.

### [Table 3]

TABLE 3

| | Example 1A | Example 1B |
|---|---|---|
| | (invention) | (invention) |
| Composition | | |
| Organopolysiloxane | 70% A2 + 30% A3 | 70% A2 + 30% A3 |
| Photoinitiator | 6.6 mmol B2 | 6.6 mmol B3 |
| Results | | |
| Solubility after 1 h in | Yes | No |
| oscillating roller mixers | | |
| Solubility after 12 h in | Yes | Yes |
| oscillating roller mixers | | |

These results show that the photoinitiators according to the invention are soluble in silicone compositions.

### Example 2: Monitoring the Polymerization of the Acrylic Functional Groups of Acrylic Silicones Under UV Mercury Lamp

The preparations were carried out as follows: the photoinitiator was weighed and introduced into the organopolysiloxane A1, and the whole was stirred until a homogeneous product was obtained (approximately 30 minutes). The mixtures were made on a basis of 2 g organopolysiloxane A1. The data are expressed in % by weight. The compositions are shown in Table 3 below.

The preparations thus obtained were then cured under UV radiation with a Mercury-Xenon lamp with reflector, at 365 nm. The power of the UV lamp was set at 510 mW·cm⁻².

Manipulations were carried out under air or in laminated conditions in order to avoid any inhibition of the reactive species by oxygen. When manipulations are carried out in “laminated” conditions, the formulation is placed between two sheets of polypropylene, then between two CaF2 discs.

Polymerization kinetics are monitored using Real-Time Fourier Transform Infra-Red (RT-FTIR, Vertex 70 from Brucker Optik). This spectroscopic technique consists of exposing the sample simultaneously to light and to an infrared ray in order to track changes in the IR spectrum at 1636 cm 1 which is a band characteristic of the C═C bond of acrylic functional groups.

The rate of conversion from C═C to C—C during polymerization is directly related to the decrease in area under the peak at 1636 cm⁻¹ calculated according to the following equation: conversion (%)=(A0−At)/A0×100 where A0 is the area under the peak before irradiation and At is the area under the peak at each time t during irradiation.

The time plot enables access to the final conversion rate, but also to other important parameters such as the maximum conversion speed ((Rp/[M]0)×100). The latter is determined by the slope of the Conversion (%)=f(t) curve at its inflection point.

The results are presented in Table 4 below.

TABLE 4

| | | Example 2A | Example 2B |
|---|---|---|---|
| | | (comparative) | (invention) |
| Composition | | | |
| Organopolysiloxane | | 99% A1 | 99% A1 |
| Photoinitiator | | 1% B1 | 1% B2 |
| Results | | | |
| 510 mW/cm² air | Rp/[M]0 × 100 | 0.1 | 43.8 |
| | Conversion (%) | 7 | 82.7 |
| 510 mW/cm² | Rp/[M]0 × 100 | 3.6 | 61.7 |
| laminated | Conversion (%) | 87.2 | 99 |

These results show that the type I photoinitiators according to the invention are more efficient in terms of conversion and reaction kinetics than the commercially available photoinitiators.

### Example 3: Evaluating the Effectiveness of Type I Photoinitiators by Monitoring the Polymerization of the Acrylic Functional Groups of Silicones in the Application of Thin Layers for a Non-Stick Application

In the following examples, silicone compositions according to the invention were coated on flexible substrates and then cured by exposure to radiation. The release performance of the substrates thus obtained was evaluated. For this purpose, the formulations were prepared as follows: a mixture of 100 parts by weight is prepared, comprising 70 parts by weight of organopolysiloxane A2 and 30 parts by weight of organopolysiloxane A3. To this mixture is then added 6.6 mmol photoinitiator B1, B2, or B3 (respectively corresponding to about 1.5 parts by weight of photoinitiator B1, 2.5 parts by weight of photoinitiator B2, and 1.8 parts by weight of photoinitiator B3). After complete solubilization of the photoinitiator, the compositions are coated onto various substrates using a Mayer rod, under the conditions described in the various examples.

Tests Carried Out on Substrates Coated with Silicone Release Coatings

Deposit: Verification of the silicone deposit coated on the surface, by X-ray fluorescence analysis of the silicon (Lab-X 3000 from Oxford). An X-ray tube excites the electron shell of the silicon atoms, which causes an emission of X-rays that is proportional to the amount of excited silicon. This value or number of counts is converted by calculation (using the standard curve) into the amount of silicone.

Smear: Qualitative verification of surface polymerization by the finger smear method which consists of:
Placing the sample of silicone-coated substrate to be checked on a flat and rigid surface;
Drawing a line with the fingertip while pressing moderately but distinctly; and
Examining the line thus made by eye, preferably in oblique light. One can thus see the presence of a finger mark, even very slight, by a difference in the gloss of the surface.
   The assessment is qualitative. “Smear” is quantified by the following notations:
A: very good, no line left by finger
B: slightly less good, barely visible line
C: clear line
D: very clear line and oily appearance of the surface, product barely polymerized i.e. a grade from A to D, from best result to worst.

Rub-off: Verifying the ability of the silicone to adhere to the flexible substrate by rubbing back and forth with the finger, which consists of:
Placing the sample of silicone-coated substrate to be checked on a flat and rigid surface, the silicone being on the upper side;
Rubbing the fingertip back and forth 10 times (over a length of about 10 cm) while pressing moderately but distinctly;
Visually examining the appearance of the rubbed area. Rub-off corresponds to the appearance of a fine white powder or small balls which roll under the finger.
   The assessment is qualitative. Rub-off is quantified by the following notations:
10: very good, no rub-off after 10 rubs back and forth
1: very bad, rub-off at the first rub
   The score corresponds to the number of rubs back and forth (from 1 to 10) at which rub-off appears,
   i.e. a score of 1 to 10, from the weakest to the best result.

Dewetting: Assessment of the degree of polymerization of the silicone layer by evaluating the transfer of silicone onto an adhesive brought into contact with the coating, using a standardized surface tension test ink. The method is as follows:
Select a sample of about 20×5 cm of the silicone-coated paper to be characterized, taken in the direction in which it unwinds (machine direction);
Cut a length of approximately 15 cm of adhesive tape, then place it with adhesive side down on the paper to be checked, without creases, exerting pressure 10 times by sliding a finger along the length of the adhesive tape. (3M “Scotch” tape, reference 610, width: 25 mm);
Remove the adhesive tape and lay it flat, adhesive side up;
Using a (disposable) cotton swab, place, on the adhesive part of the tape, a line of ink extending for a length of approximately 10 cm (SHERMAN or FERARINI and BENELI brand of inks with a surface tension of approximately 30 dyn/cm and a viscosity of 2 to 4 mPa/s). Immediately start the timer;
Entry into the phase of the dewetting phenomenon is considered to have occurred when the line of ink changes its appearance: then stop the timer;
Application of the ink onto the adhesive part of the tape must take place within 2 minutes after the coating with silicone;
If the obtained result is <10 seconds, it is considered that migration of silicone onto the adhesive has occurred and that polymerization is not complete;
A score of 0 to 10 will be given, corresponding to the time elapsed in seconds before the dewetting phenomenon is observed;
If the obtained result is 10 seconds, polymerization is considered to be complete. In this case, a score of 10 is given, meaning that the result is very good;
Note down the score obtained and the ink used (name, brand, surface tension, viscosity).

Extractables: Measurement of the amount of silicone that is not grafted to the network formed during polymerization. These silicones are extracted from the film by immersing the sample, as soon as it leaves the machine, into MIBK (methyl isobutyl ketone) for a minimum of 24 hours. This is measured by flame atomic absorption spectroscopy.

### Preparation of Self-Adhesive Multilayer Items

A TESA 7475 standard adhesive substrate (substrate=PET-adhesive=acrylic) is laminated to the silicone liner produced above (=substrate coated with a silicone coating obtained by curing under UV) in order to form a multilayer item. Tensile tests are carried out in order to determine the release forces before and after aging, as well as the subsequent adhesion and loop-tack values. These tests are described below.

### Test Carried Out on the Multilayer Items Obtained

Subsequent adhesion (or “SubAd”): Measurement verifying the residual tack of adhesives (TESA 7475) having been in contact with the silicone coating, according to the FINAT 11 (FTM 11) test known to those skilled in the art. Here the reference test specimen is PET, and the adhesives remained in contact with the silicone surface to be tested for 1 day at 70° C.

The results are expressed in % residual adhesive force of the reference tape:

CA=(Fm2/Fm1)×100 as a%,

with:
Fm2=Average tape release force after contact with silicone substrate for 20 h; and
Fm1=Average tape release force without contact with silicone substrate.
   Adhesion above 90% is desired.

Release forces: The peeling force measurements are carried out with the TESA 7475 standard adhesive. The test specimens of the multilayer item (adhesive in contact with the silicone surface) were kept for 1 day at 23° C., and 1 day at 70° C. under the pressure conditions required according to the FINAT 10 test, then tested at low peeling speed according to the FINAT 3 test (FTM 3) known to those skilled in the art.

The release force is expressed in cN/inch and is measured using a dynamometer, after pressurization of the samples either at room temperature (23° C.) or at higher temperature for accelerated aging tests (usually 70° C.).

The tested formulations and the test results are shown in Table 5 below.

TABLE 5

| | | |
|---|---|---|
| Example 3A | Example 3B | Example 3C |
| (comparative) | (invention) | (invention) |

Composition

| | | | |
|---|---|---|---|
| Organopolysiloxane | 70% A2 + | 70% A2 + | 70% A2 + |
| (100 parts by weight) | 30% A3 | 30% A3 | 30% A3 |
| Photoinitiator (mmol of | 6.6 mmol B1 | 6.6 mmol B2 | 6.6 mmol B3 |
| photoinitiator added) | | | |

Results

| | | | |
|---|---|---|---|
| Smear | B | A | A |
| Rub-off | 10 | 10 | 10 |
| Dewetting | 5 | 10 | 10 |
| Extractables (%) | 9.6 | 5.35 | 5.6 |
| Release forces | 8.9 | 7.3 | 6.7 |
| TESA 7475 1 day 23° C. | | | |
| (cN/inch) | | | |
| Release forces | 9.6 | 9.1 | 7.8 |
| TESA 7475 1 day 70° C. | | | |
| (cN/inch) | | | |
| Subsequent adhesion | 74 | 96 | 95 |
| 1 day 70° C. (%) | | | |

The results for the smear, rub-off, and dewetting for type I photoinitiators according to the invention indicate good polymerization of the acrylic silicone formulation. This good polymerization is also reflected in the low level of extractables. The films obtained have the expected non-stick properties. In particular, the subsequent adhesion is better than that of the comparative example.

Thus, the type I photoinitiators according to the invention can be used to produce release systems.

1. Radiation-curable silicone composition X comprising:
a. at least one organopolysiloxane A including at least one (meth)acrylate group;
b. at least one radical photoinitiator B, which is a compound of formula (I) 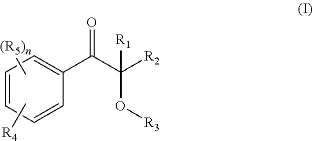
in which
R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
R₃ is H or a C₁-C₆ alkyl group;
R₄ is a 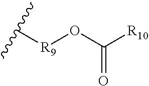
group;
each R₅ group independently represents a C₁-C₆ alkyl group;
n=0, 1, 2, 3, or 4;
R₉ is a C₁-C₆ alkylene group or a C₁-C₆ heteroalkylene group; and
R₁₀ is a linear or branched C₁-C₁₈ alkyl group.

2. Silicone composition X according to claim 1, wherein the compound of formula (I) is a compound of formula (II) 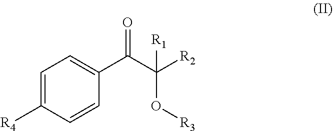
in which
R₁ and R₂ are chosen, independently of each other, from C₁-C₆ alkyl groups and C₃-C₇ cycloalkyl groups:
or R₁ and R₂, together with the carbon atom to which they are attached, form a C₃-C₇ cycloalkyl group;
R₃ is H or a C₁-C₆ alkyl group; and
R₄ is a 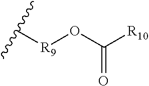
group.

3. Silicone composition X according to claim 1, wherein the compound of formula (I) is a compound of formula (III) 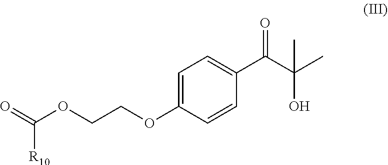
in which R₁₀ is a linear or branched C₁-C₁₈ alkyl group.

4. Silicone composition X according to claim 1, wherein the organopolysiloxane A comprises:
a) at least one unit of the following formula (IV):
RₐZ_{b}SiO_{(4-a-b)/2} (IV)
a formula in which:
the R symbols, which are identical or different, each represent a linear or branched C₁ to C₁₈ alkyl group, a C₆ to C₁₂ aryl or aralkyl group, which is possibly substituted,
the Z symbols are monovalent radicals of formula -y-(Y′)n in which:
y represents a polyvalent C₁-C₁₈ alkylene or heteroalkylene group, said alkylene and heteroalkylene groups able to be linear or branched, and possibly being interrupted by one or more cycloalkylene groups, and possibly being extended by bivalent oxyalkylene or polyoxyalkylene radicals at C₁ to C₄, said alkylene, heteroalkylene, oxyalkylene, and polyoxyalkylene groups possibly being substituted by one or more hydroxy groups,
Y′ represents a monovalent alkenylcarbonyloxy group, and
n is equal to 1, 2, or 3, and
a is an integer equal to 0, 1, or 2, b is an integer equal to 1 or 2, and the sum a+b=1, 2, or 3; and
b) optionally, units having the following formula (V):
RₐSiO_{(4-a)/2} (V)
a formula in which:
the R symbols are as defined above in formula (IV), and
a is an integer equal to 0, 1, 2, or 3.

5. A method for preparing a silicone elastomer capable of being used as a release coating on a substrate, said method using the silicone composition X according to claim 1.

6. Silicone elastomer obtained by curing a silicone composition X according to claim 1.

7. Method for preparing a coating on a substrate, comprising the following steps:
applying a silicone composition X according to claim 1, and
curing said composition by electron or photon irradiation.

8. Coated substrate obtainable by the method according to claim 7.

9. A method for preparing silicone elastomer items by an additive manufacturing process using the composition X according to claim 1.

10. Compound of formula (I) 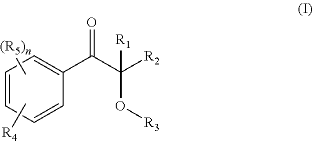
in which
R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
R₃ is H or a C₁-C₆ alkyl group;
R₄ is a 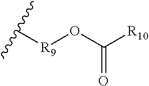
group;
each R₅ group independently represents a C₁-C₆ alkyl group;
n=0, 1, 2, 3, or 4;
R₉ is a C₁-C₆ alkylene group or a C₁-C₆ heteroalkylene group; and
R₁₀ is a linear or branched C₁-C₁₈ alkyl group.

11. Compound according to claim 10, characterized in that it is of the following formula (II) 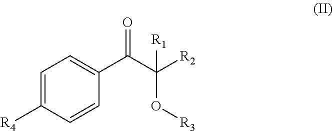
in which
R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
R₃ is H or a C₁-C₆ alkyl group; and
R₄ is a 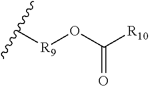
group.

12. Compound according to claim 10, characterized in that it is of the following formula (III) 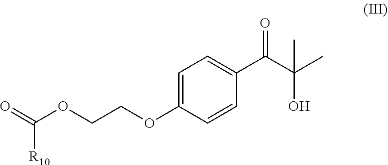
in which R₁₀ is a linear or branched C₁-C₁₈ alkyl group.

13. A method for curing a radiation-curable composition Y comprising at least one radiation-curable unsaturated compound D, said method using a compound according to claim 10 as a radical photoinitiator.

14. The method according to claim 13, wherein the radiation-curable unsaturated compound D is a hydrocarbon compound comprising one or more double bonds, and, optionally, one or more heteroatoms chosen among N, P, O, S, and F.

15. The method according to claim 13, wherein the radiation-curable unsaturated compound D is an organopolysiloxane comprising one or more double bonds.

16. A method for curing a radiation-curable composition Y comprising at least one radiation-curable unsaturated compound D, said method using a compound of formula (I) as radical photoinitiator 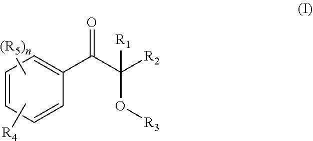
in which
R₁ and R₂ are chosen, independently of each other, from C₁-C₆ alkyl groups and C₃-C₇ cycloalkyl groups;
or R₁ and R₂, together with the carbon atom to which they are attached, form a C₃-C₇ cycloalkyl group;
R₃ is H or a C₁-C₆ alkyl group;
R₄ is a 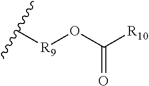
group;
each R₅ group independently represents a C₁-C₆ alkyl group;
n=0, 1, 2, 3, or 4;
R₉ is a C₁-C₆ alkylene group or a C₁-C₆ heteroalkylene group; and
R₁₀ is a linear or branched C₁-C₁₈ alkyl group and said unsaturated compound D being an organopolysiloxane comprising one or more double bonds.
